(19) United States
(12) Patent Application Publication
(10) Pub. No.: US 2014/0194321 A1
(43) Pub. Date: Jul. 10, 2014

(54) CELL-BASED METHOD TO DETECT SKIN SENSITIZERS

(71) Applicant: CELLOMICS, INC., Pittsburgh, PA (US)

(72) Inventor: Monica Jo Tomaszewski, Pittsburgh, PA (US)

(73) Assignee: CELLOMICS, INC., Pittsburgh, PA (US)

(21) Appl. No.: 13/734,751

(22) Filed: Jan. 4, 2013

(51) Int. Cl. C12Q 1/02

(52) U.S. Cl. CPC C12Q 1/025; G06F 19/10
USPC 506/10; 506/39; 702/19

(57) ABSTRACT

A method of predicting whether a compound is a skin sensitizer using an in vitro approach. The method includes a step of imaging immune effector cells positioned within a plurality of containers to obtain imaged cellular targets, each container being treated with a different concentration of a compound. The method further includes a step of quantitatively measuring the imaged cellular targets over a range of concentrations of the compound to detect changes in multiple cellular targets of the immune effector cells associated with skin sensitivity. The method further includes a step of analyzing measurements obtained from the measured imaged cellular targets over the range of concentrations of the compound to determine whether the compound is a skin sensitizer.

### BACKGROUND OF THE INVENTION

1. The Field of the Invention

The present invention relates to methods, systems, and computer program products for determining if a compound is a skin sensitizer. More specifically, the present invention relates to using in vitro cells and cell-based assay methods to determine if compounds are skin sensitizers.

2. The Relevant Technology

Allergic contact dermatitis (ACD) is a detrimental health effect that can develop in those exposed to skin sensitizing chemicals and products that contain the chemicals. To decrease the occurrence of this adverse reaction, U.S. Food and Drug Administration (FDA) regulations require that testing be performed to identify chemicals that are responsible for this effect. Products that contain the skin sensitizing chemicals can then be labeled accordingly.

Historically, the use of animals has been required for testing chemicals for the potential for skin sensitization in the U.S. and the E.U. The currently accepted method of testing is the local lymph node assay (LLNA). The LLNA measures cell proliferation in the draining lymph node of a test animal as a measure of skin sensitization. The cell proliferation is measured via radiolabeled cells, following dermal exposure to the compounds during the induction phase of sensitization. Many live animals, typically mice, are required to perform the LLNA for each chemical compound tested.

Recently, a modified version of the LLNA (known as the revised LLNA or rLLNA) was developed that uses a chemical method of measuring cell proliferation and decreases the animals required for the testing of each chemical compound.

Although the LLNA and the rLLNA are able to determine the skin sensitivity of compounds that are tested, both the LLNA and the rLLNA suffer from significant drawbacks. They are time and labor intensive and must use a number of animals to test each chemical compound. And because many consumer chemical compounds must be tested each year for skin sensitivity, a significant number of animals are required. Not only is it expensive to house and maintain the animals, but the chemicals often harm or even disfigure the animals. In addition, despite the LLNA being used as the standard testing method, discrepancies have been found between results of the LLNA and previously used guinea pig tests as well as human data.

In light of the above, providing a simple, efficient, in vitro method of testing for skin sensitizers is of particular relevance to consumer product safety and occupational health.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope. In the drawings, like numerals designate like elements. Furthermore, multiple instances of an element may each include separate letters appended to the element number. For example two instances of a particular element “20” may be labeled as “20a” and “20b”. In that case, the element label may be used without an appended letter (e.g., “20”) to generally refer to every instance of the element; while the element label will include an appended letter (e.g., “20a”) to refer to a specific instance of the element.

FIG. 1 is a perspective view of an imaging system incorporating features of the present invention;

FIG. 2 is a flow diagram illustrating a method for determining if a compound is a skin sensitizer;

FIG. 3 is a perspective view of a 96-well plate used in various embodiments of the present invention;

FIG. 4 illustrates an exemplary setup or map for the wells of a 96-well plate;

FIG. 5 is a flow diagram illustrating one embodiment of a method for imaging immune effector cells;

FIG. 6 is a flow diagram illustrating one embodiment of a method for analyzing measurements obtained from measured imaged cellular targets over a range of concentrations of a compound to determine whether the compound is a skin sensitizer;

FIGS. 7A-7C illustrate different types of dose response curves;

FIG. 8 illustrates a decision matrix according to one embodiment;

FIG. 9 is a table containing photographed images of U937 cells, the top, middle, and bottom rows showing cells treated with a vehicle, a known sensitizer, and a compound of interest, and the columns showing cells stained with specific fluorescent dyes to detect, from left to right, nuclei, whole cells, and CD54 levels;

FIGS. 10A and 10B illustrate dose response curves obtained for U937 cells treated with Hexylcinnaldehyde; and

FIG. 11 is a flow diagram illustrating an embodiment of a method of screening a plurality of compounds for skin sensitivity.

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein. It will also be understood that any reference to a first, second, etc. element in the claims or in the detailed description, is not meant to imply numerical sequence, but is meant to distinguish one element from another unless explicitly noted as implying numerical sequence.

In addition, as used in the specification and appended claims, directional terms, such as “top,” “bottom,” “up,” “down,” “upper,” “lower,” “proximal,” “distal,” “horizontal,” “vertical,” and the like are used herein solely to indicate relative directions and are not otherwise intended to limit the scope of the invention or claims.

The basic premise of animal based sensitizing assays is that the assays ultimately produce proliferation in immune cells of the animal, specifically to the draining lymph node from the site of treatment. The LLNA is a simple test that enumerates these proliferating cells. However, the test is costly due to the labor required to perform the assay and house the animals, the disposal of radioactive markers (if used), and the time to analyze the results. Furthermore, the use of live animals for chemical testing is frowned upon by the general public. In light of the above, there is a concerted effort to develop assays that replace animal testing, are high throughput, and do not lose any information in the transition from in vivo to in vitro.

To this end, multiple labs have attempted to devise a method of obtaining similar results to the LLNA via a flow cytometric approach using either primary cells or cell lines. Similar to the LLNA, these assays produce a population-based result. However, instead of using cell numbers, as in the LLNA, the flow cytometric assays analyze the production of particular proteins in the treated cells over a background. While the ultimate outcome of the LLNA is based on cell counts, the LLNA also implicitly identifies that cells travel e.g., from the ear to the lymph node of the mouse that is used for testing. Because flow cytometry methodology lacks a functional aspect, the cytometric assay is simply unable to replicate this aspect of the LLNA.

In contrast, embodiments of the present invention are directed to methods, systems, and computer program products for determining skin sensitivity of compounds using an in vitro assay that replicates the in vivo effects of a skin sensitizer. These effects, which are quantitatively measured in embodiments of the present invention, can include differentiation or maturation with protein production, an increase of cell number/proliferation, and cell trafficking or motility.

Compared to conventional methods (e.g., the LLNA and rLLNA), determination of skin sensitivity according to embodiments of the present invention can be performed in less time, is less labor intensive, and does not require the use of live animals. In addition, unlike in the LLNA and the rLLNA, aspects of the overall skin sensitization pathway can be quantified, and this quantification be done in a cell line based format.

In addition, the collected cell number and morphology data can indicate concentrations at which the chemicals are toxic, allowing adjustments to be made so that determination of the chemical's skin sensitizing potential can be done at lower non-toxic concentrations.

Embodiments of the invention can employ the use of a high-content screening (HCS) system. In a high content screening assay, the processes discussed herein can be more efficiently recapitulated due to the ability to deal with spatial characterization as well as cell enumeration. As a result, information can be quickly and easily obtained for a range of concentrations for a compound.

Thus, embodiments of the present application can apply physical and functional changes over a range of concentrations to a decision tree that predicts sensitivity. Because the LLNA is itself a functional test, embodiments of the present invention provide the best opportunity for an in vitro replacement to the LLNA.

The innovative processes presented herein enable the prediction of skin sensitizing potential for chemical compounds with high specificity and sensitivity (i.e., low false positive and negative rates, respectively).

Although the discussion set forth herein is directed to using the innovative processes for predicting whether a compound is a skin sensitizer, it will be appreciated that the processes can also be used to determine other types of sensitivity. By way of example and not limitation, embodiments of the invention can also be used to determine whether other parts of the body, such as the mouth, throat, stomach, and lungs, among others are sensitive to particular compounds. Sensitivity potential of a compound used in conjunction with non-chemical insults, such as UV-radiation, can also be determined.

Embodiments of the present invention may comprise or utilize a special purpose or general-purpose computer including computer hardware, such as, for example, one or more processors, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: computer storage media and transmission media.

Computer storage media includes RAM, ROM, EEPROM, CD-ROM or other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired and wireless) to a computer, the computer properly views the connection as a transmission medium. Transmission media can include a network and/or data links which can be used to carry data or desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to computer storage media (or vice versa). For example, computer-executable instructions or data structures received over a network or data link can be buffered in RAM within a network interface module (e.g., an “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions comprise, for example, instructions and data which cause a general purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, traffic sensors, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

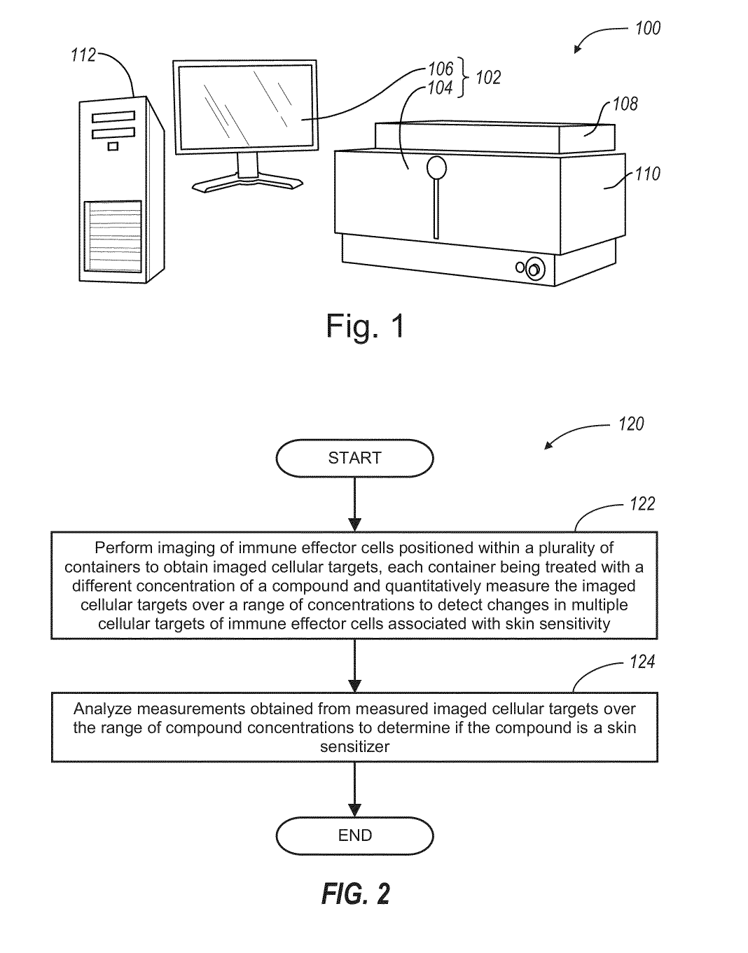

Accordingly, in this specification and in the following claims, a computer system is also defined to include imaging systems (e.g., imaging system 102 in FIG. 1).

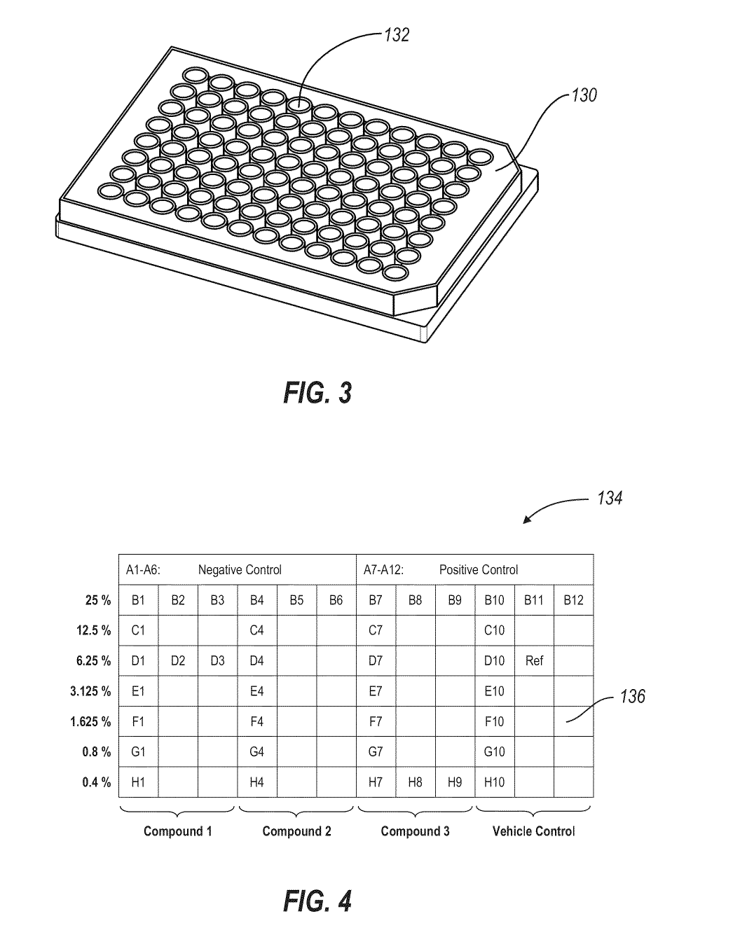

FIG. 1 illustrates an exemplary system 100 incorporating features of the present invention. At the heart of the system is a quantitative high-content cell imaging system 102 in which cells are scanned and analyzed. The exemplary cell imaging system 102 includes, but is not limited to, an imaging device 104 with a user display device 106. Imaging device 104 generally includes a stage housing 108 mounted on a microscope assembly 110 having a plurality of objectives. Stage housing 108 is configured to house the components required to position a specimen plate (such as, e.g., 96-well plate 130 shown in FIG. 3) or a slide containing cells so microscope assembly 110 can image the cells using the objectives to allow high content screening of the cells to be performed, as is known by one skilled in the art. Analyzing and storing of the data obtained from the imaging can be performed by imaging device 104 with results being displayed to the user on user display device 106. Examples of commercially available high content cell imaging systems that can be used are the Thermo Scientific® ToxInsight® IVT Platform manufactured by Cellomics, Inc. of Pittsburgh, Pa., a subsidiary of Thermo Fisher Scientific, Inc. and the Thermo Scientific® ArrayScan® VTI HCS Reader, also manufactured by Cellomics, Inc. Other cell imaging systems can also be used, including cell imaging systems that are non high-content imaging systems.

System 100 can also include an external computing device 112, if desired. External computing device 112 can comprise a general purpose or specialized computer or server or the like, as defined above. External computing device 112 can be used as a controller for the system as well as for performing, by itself or in conjunction with imaging device 104, the analyzing and/or storing of the data obtained by imaging device 104. In some embodiments, external computing device 112 can also display results to the user on user display device 106. External computing device 112 can communicate with imaging device 104 and/or display device 106 directly or through a network, as is known in the art.

In one embodiment of the invention, one or more of the method steps described herein are performed as a software application. However, the present invention is not limited to this embodiment and the method steps can also be performed in firmware, hardware or a combination of firmware, hardware and/or software. Furthermore, the steps of the methods can exist wholly or in part on imaging device 104, external computing device 112, and/or other computing devices.

An operating environment for the devices of the system may comprise or utilize a processing system having one or more microprocessors and system memory. In accordance with the practices of persons skilled in the art of computer programming, the present invention is described below with reference to acts and symbolic representations of operations or instructions that are performed by the processing system, unless indicated otherwise. Such acts and operations or instructions are referred to as being “computer-executed,” “CPU-executed,” or “processor-executed.”

In embodiments of the present invention, skin sensitizing compounds can be identified via the induction of cell surface protein production along with functional chemotaxis assays that can also provide toxicity information for the compounds. Using the data from the biological assays, a prediction can be made as to whether a compound is a sensitizer or a non-sensitizer. In addition, the toxicity of the compound at the tested concentrations can be determined and, if toxic, a recommendation for additional testing of the compound for sensitization at non-toxic doses can be made.

The assay process can consist of several parts. A first part can include a specific manner of treating and fluorescently labeling cells of haemopoetic origin, and quantitatively measuring several imaged cellular properties, functions and/or targets (collectively referred to herein as “targets”) to detect the initiation of skin sensitizing potential. In one embodiment, cell lines derived from dendritic cells are used. A second part can include analyzing and correlating the quantitatively measured data (i.e., the targets) acquired in the first part to determine if the compound of interest is a skin sensitizer.

FIG. 2 illustrates an assay method 120 for determining if a compound is a skin sensitizer according to one embodiment. A first step 122 of the exemplary process includes imaging of immune effector cells and measuring cellular targets obtained from the imaging. Specifically, in step 122 imaging of immune effector cells positioned within a plurality of containers is performed to obtain imaged cellular targets, each of the containers having been treated with a range of concentrations of a compound. The imaged cellular targets are also quantitatively measured in step 122 to detect changes in multiple cellular targets of the immune effector cells associated with skin sensitivity.

In many of the examples discussed herein, the cellular targets monitored in the assays are cell gain/loss, specific cell markers, and motility of the cells; however, other cellular properties, functions and/or targets can also be monitored.

In a second step 124 of the exemplary process, the quantitative multiparametric cellular target data acquired in step 122 is analyzed over the range of compound concentrations to determine if the compound of interest is a skin sensitizer.

The exemplary skin sensitization detection and analysis process 120 enables the systematic investigation of the effects a compound has on immune effector cells and accurately predicts therefrom the skin sensitivity of the compound with high specificity and sensitivity (i.e., low false positive and negative rates respectively).

To accomplish step 122, an assay can be performed on immune effector cells treated with various concentrations of a compound of interest, then the responses of those cells can be measured. In some embodiments of the invention, a multi-well microplate can be used.

An example of a multi-well microplate that can be used is well plate 130, shown in FIG. 3. As depicted, well plate 130 has ninety-six individual wells 132 arranged eight rows and twelve columns. Other sized well plates, as are known in the art, can alternatively be used. Well plate 130 can have a transparent bottom to facilitate imaging or other detection methods, if desired. Cells can be treated in separate wells 132 for a specific period of time with i) a vehicle (e.g., DMSO or buffer) to be able to provide normalized data, ii) sample compounds where the skin sensitivity of the compounds are desired to be known, iii) a known skin sensitizer (used as a positive control), and iv) a known skin non-sensitizer (used as a negative control).

For example, FIG. 4 illustrates an example plate setup or map 134 that can be used for well plate 130. In plate map 134, each cell 136 represents a different well 132 of plate 130. As noted above, in a standard 96-well plate, the wells are arranged in eight rows and twelve columns, which is mirrored in plate map 134. For ease of use, the rows are lettered and the columns are numbered. Thus, “B4”, e.g., represents the second row and the fourth column. According to the example plate map 134, each well plate 130 can include samples corresponding to three separate compounds of interest (denoted “Compound 1,” “Compound 2,” and “Compound 3” in FIG. 4). For each compound, different concentrations or doses can be tested concurrently, as shown. Each well plate 130 can also include samples corresponding to a known skin sensitizer (denoted “Positive Control”) and a known skin non-sensitizer (denoted “Negative Control”).

Wells can also be associated with vehicle control samples (denoted “Vehicle Control” in FIG. 4). For example, one or more of the wells (e.g., well “Ref” on FIG. 4) can be used to measure the optimal exposure time for each assay target. Other wells can be used to determine the amount of exposure the plate receives. The exposure values from each plate can then allow a normalization to be computed between plates, as is known in the art. Vehicle control samples can be made either in serial dilutes or in a fixed vehicle concentration (e.g., 1% DMSO). It is appreciated that plate map 134 is exemplary only; other plate maps can alternatively be used.

The indicators of skin sensitization can be classified in three types of cellular response: i) gross markers, ii) specific markers, and iii) functional outcomes. Any of these can be measured and quantified in the immune effector cells (e.g., U937) treated with compounds in embodiments of the present invention.

Gross markers are used to identify both total and live numbers of cells. Cells can be identified by the fluorescence staining of a major cellular structure, such as by a nuclear or a whole cell dye, and then counted to give the number of cells. This cell number can provide an indication of toxicity of the compound via cell loss. The cell number can be measured via manual counts (albeit labor intensive), flow cytometry, image analysis and plate reader methodologies. In addition to providing cell numbers, imaging and flow cytometry also have the ability to identify the shape of the nucleus or cell body to provide additional toxicity data.

Specific markers are indicative of specific cellular changes, identified by changes in specific cellular targets, induced by the presence of the compound. These specific markers can be cell-type specific and have a role in the differentiation of the cell type that is induced by sensitization. In skin sensitization, markers on U937 cells that are associated with differentiation, e.g., CD54, CD80, CD86, HLA-DR, can be quantified. In many embodiments discussed herein, CD54, an up-regulated marker, is used as the specific marker of U937 that is quantified. However, other cellular molecules can also be detected on the cell surface, either instead of, or in addition to CD54. The measurement of specific markers can also be detected using different detection methods and processes.

Since specific markers are usually cell surface protein molecules that are either translocated from inside the cell or from new protein production, the ability to detect specific markers can follow several forms. For instance, the proteins can be detected by immunofluorescence, by a fluorescent ligand, or with a specialized subclone of the cell type in question that produces a fluorescent variant of the molecule. Automated imaging and analysis or flow cytometry can be used to detect these changes.

Functional outcomes indicate functional responses induced by exposure to the compound. Because the LLNA is a functional test, as discussed above, using functional outcomes helps embodiments of the present invention to replicate the in vivo responses in an in vitro environment. The ability for cells to demonstrate a functional response to treatment of the compound can help to determine how sensitization compounds influence the differentiation of a cell line. Functional outcomes are typically measured using a chemotactic approach, as discussed in more detail below. Chemotaxis is the movement of cells in the direction of a chemical gradient and is useful for measuring many functional outcomes, such as, e.g., cell motility. Virtually any method of measuring movement in response to a compound that induces chemotaxis can be a valid measurement technique.

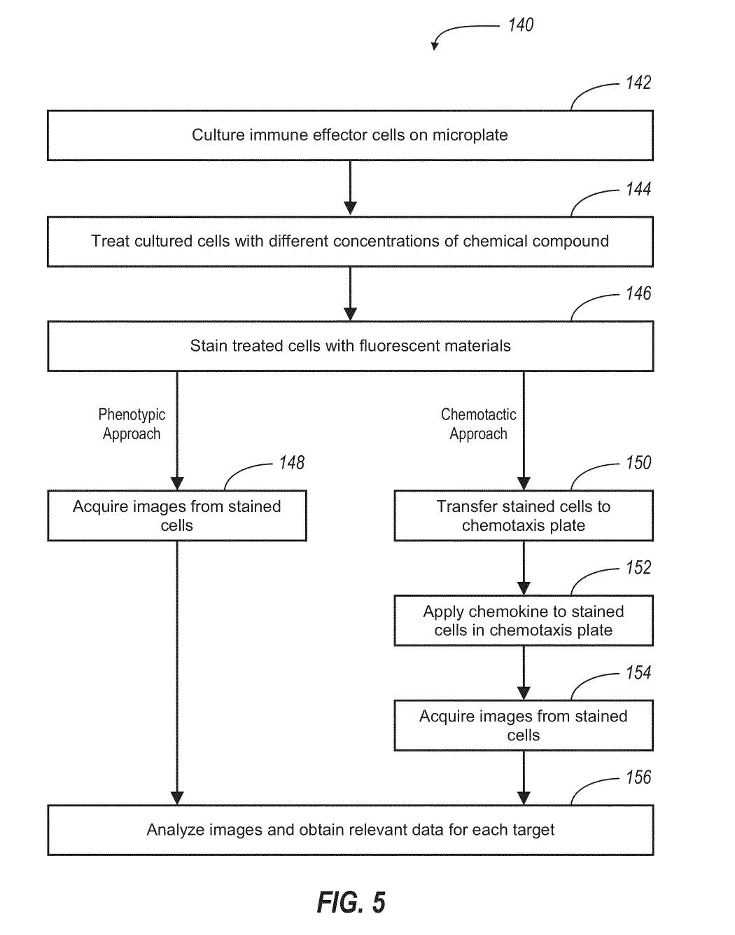

FIG. 5 illustrates one embodiment of a method 140 for accomplishing step 122 of method 120 (FIG. 2) using well plate 130. Method 140 includes steps 142-156, which are discussed below.

In step 142, immune effector cells are cultured on a microplate, such as well plate 130. In one embodiment, the area of interest is the skin; therefore the assay can be performed using U937 cells as the immune effector cells. Other types of cells can also be used depending on the body area for which sensitivity is being investigated.

U937 cells are of histocytic origin and mimic skin-resident Langherhans Cells (LCs) which are some of the first immunological cells that come in contact with a skin sensitizer in live beings. In particular, U937 cells mature from a monocytic to an LC-like phenotype after being exposed to sensitizers. U937 cells can be grown and cultured on multi-well microplates in optimal cell growth media,

In step 144, the cultured cells are treated with various concentrations of the compound of interest. Many cellular targets exhibit different responses at different doses of different compounds. For example, a cellular target may show its skin sensitivity response to a particular chemical at an intermediate concentration but may be toxic at a higher concentration and show no response at lower concentrations. To take these conditions into account, many of the embodiments disclosed herein simultaneous monitor responses of multiple cellular targets over a range of compound concentrations.

To do so, each well of cells in the microplate can be treated with a different concentration of the compound so that a range of cellular dose responses for each cellular target is obtained for the compound. For example, in the exemplary plate map 134 of FIG. 4, each compound has samples corresponding to a range of concentrations between 0.4% and 25%. It will be appreciated that this range is exemplary only; other ranges can also be used, depending on the compounds tested. Furthermore, the highest concentration values of each compound can be as high as the peak serum concentration of the compound.

To take into account sample variations, a plurality of wells can be used for each concentration level of a compound to generate multiple samples for the concentration level. For example, in the exemplary plate map 134 of FIG. 4, wells B1, B2, and B3 correspond to three separate samples of Compound 1 at a 25% concentration level and wells D1, D2, and D3 correspond to three separate samples of the same compound at a 6.25% concentration level. If desired, the number of samples can be reduced if the sample variation across the plate is low.

In step 146, after being treated with the various concentrations of the compound, the treated cells are stained with specific fluorescent materials, as is known in the art, to detect different cellular targets, such as those discussed above, whose changes are indicative of skin sensitivity. For example, in one embodiment, the cells are stained to help detect the number of cells, the CD54 intensity, and the cell motility. The cell staining procedure can be optimized to ensure the proper staining of different cellular targets. Although specific cellular targets are monitored for the exemplary embodiments discussed herein, other cellular targets can also be monitored for other sensitization assessments by using similar fluorescent probes.

At this point, one of two branches is taken depending on whether a phenotypic approach or a chemotactic approach is required. For a phenotypic approach, which involves no additional chemicals that need to be partially separated from the chemicals in the well, step 148 is next performed; for a chemotactic approach, which involves using additional chemicals partially separated from the chemicals in the well, step 150 is next performed.

In step 148, cellular targets are imaged directly from microplate 130 to acquire images of the stained cells. The fluorescently labeled cells can be detected by manual microscopy or by using a fluorescence imaging system, such as, e.g., imaging system 102 (FIG. 1). However, conducting the assay using manual microscopy would be laborious in assaying a range of concentrations for each compound. An automated high-content imaging system provides an effective option to automatically detect and quantify the targets accurately with great speed, enabling the analysis of multiple compounds, doses and conditions. Images of the cells can be acquired in distinct, different colors to detect the different fluorescently labeled cellular targets, and then stored and analyzed using image analysis programs. Furthermore, the imaging can be done simultaneously for all concentrations of a compound, yielding more accurate results. Therefore, an automated imaging system is preferred. The fluorescently labeled cells can alternatively be detected by flow cytometry.

As noted above, many functional outcomes, such as, e.g., cell motility, are measured using chemotaxis. To detect and measure these targets, steps 150-154 are followed to obtain the desired data. In step 150, the cells are transferred to a second plate or other device that can allow chemotaxis to occur. For example, after exposure to the compound, cells can be selectively stained with a viability dye to detect live cells. The stained cells can then be transferred to a particular type of microplate that is able to sustain a chemical gradient, such as, e.g., a chemotaxis plate. One embodiment of a chemotaxis plate that can be used with embodiments of the present invention is the Iuvo™ Chemotaxis Assay Plate #6006 manufactured by Bellbrook Labs of Madison, Wis. Of course, other chemotaxis plates can also be used.

In step 152, a chemotactic chemical, such as, e.g., RANTES/CCL5 or SDF-1/CXCL12, can be introduced to the second microplate as a chemokine to induce chemotaxis, and the stained cells can be allowed to migrate in response to the chemokine In many embodiments discussed herein, SDF-1 was used as the chemokine to induce chemotaxis. However, other chemokines can also be used to induce chemotaxis, such as MIP-3beta (CCL19) and CCL21.

As is known in the art, chemotaxis measurements can be performed in several manners. These can include using Boyden chamber based devices, such as, e.g., transwell inserts, that can fit in microwell plates, specifically manufactured microplates designed for chemotaxis that can sustain a chemotactic gradient (e.g., the Iuvo™ Chemotaxis Assay Plate #6006, discussed above), and simple assays where the cells are tracked during introduction of the chemokine.

The chemotaxis devices that subject the cells to a physical separation from the chemokine, may be advantageous, as those devices allow for chemicals that crystallize or fluoresce to be removed from the area of analysis. This enables a more accurate cellular count using automated methods. In many other methods, the presence of fluorescent compounds can make it difficult to identify and accurately count individual cells.

In step 154, once cell response to chemotaxis has occurred, the functional outcome can be imaged in a similar manner to that discussed above with respect to step 148.

In step 156, the acquired images from both phenotypic and chemotactic approaches can be analyzed to obtain and measure relevant data for each cellular target. Once multiple images for each condition have been acquired, different regional areas of each cell can be assigned by image analysis algorithms as are known in the art. For example, to obtain the number of cells, the nuclear region can be masked by DNA staining in the cell and a cytoplasmic area can be masked by the staining of the whole cell with the nuclear area subtracted. Cell number values can be determined by measuring the number of cells in a defined area. The amount of surface antibody staining of cells can be determined by the fluorescence that is associated with a certain region that is a defined distance away from the nuclei of the cell, but still within the area of the cell. Similar processes can be used to obtain data for other cellular targets, as is known in the art.

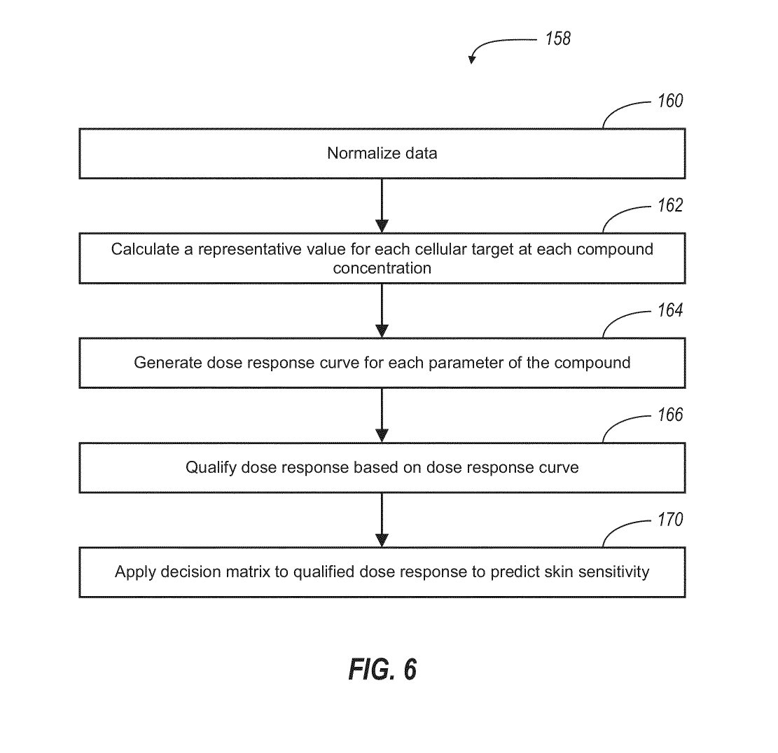

To accomplish step 124 of FIG. 2, the quantitative cellular target data obtained from the image analysis of the imaged fluorescent cells performed in step 122 can be used to make a skin sensitivity prediction having high sensitivity and specificity. FIG. 6 illustrates one embodiment of a method 158 for accomplishing step 124 of method 120 (FIG. 2) to predict whether a compound is a skin sensitizer. Method 158 includes steps 160-168, which are discussed below.

In step 160, the data corresponding to the different compound concentrations is normalized. In one embodiment, measurements generated from the vehicle-treated cell image analysis can be used. Because the vehicle-treated cells have not been treated with any of the compound, the vehicle-treated cells represent a baseline for the cellular targets. As such, a normalization of the data obtained from the compound-treated cells can be determined by subtracting the baseline response of the vehicle-treated cells from the measurements of the responses of the compound-treated cells. Measured values of the compound-treated cells that are below baseline levels can be notated as lacking a response, and can be ignored in further analysis, if desired.

In step 162, a representative value, such as, e.g., the average value, of each of the observed targets of the samples can be calculated for each concentration of the compound. This can be done by calculating a mean value for the measured target at each concentration level, taking into account all of the samples corresponding to the particular concentration level.

For example, mean values for gross markers can be computed for the number of cells using either the number of cell bodies or the number of nuclei detected. For specific markers, mean values can be computed for each of the features (e.g., CD54 (ICAM-1) intensity). To get a relative amount (or intensity) of up-regulated markers (e.g., CD54 (ICAM-1)) per cell, the per-replicate amount of the staining can be divided by the number of cells for the particular replicate. For functional outcomes, mean values can be computed for the number of live cells that demonstrate movement. Other mean values can also be used. In addition, other representative values can also be used for each concentration of the compound, such as a highest measured value, a lowest measured value, or other representative values.

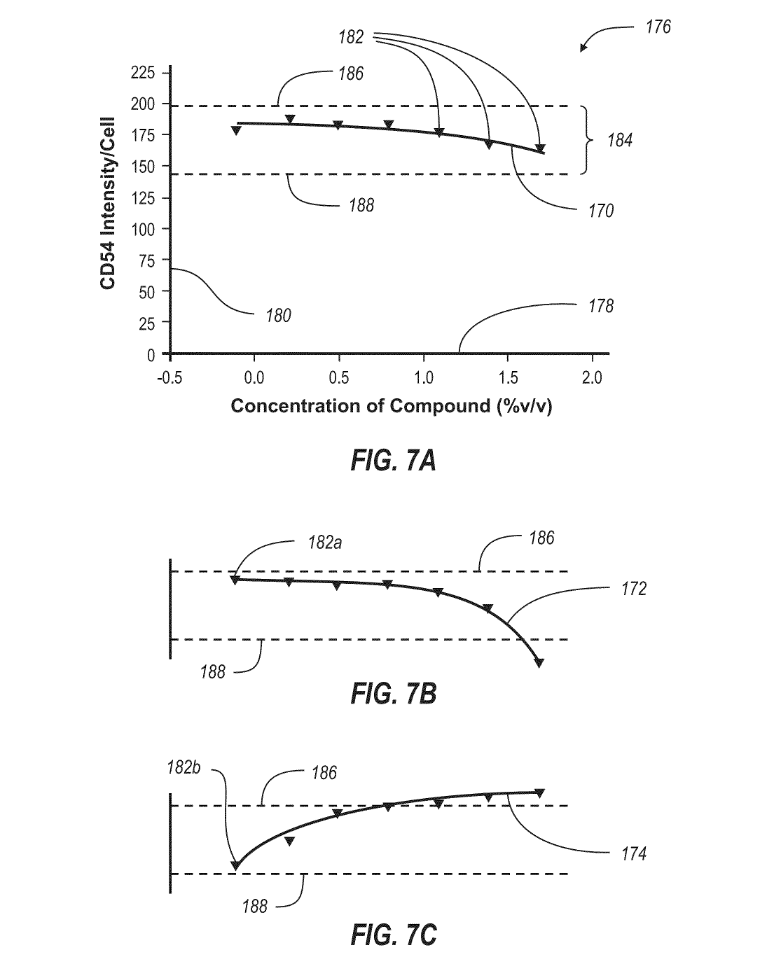

In step 164, a dose response curve is generated for each target. This can be done by plotting on a graph the average value of the target for each concentration. FIG. 7A-7C depict examples of dose response curves 170, 172, 174 according to one embodiment. In FIGS. 7B and 7C, however, much of the graph detail has been omitted for clarity sake. FIG. 7A shows a graph 176 in which the x-axis 178 represents the concentration of the compound and the y-axis 180 represents the average intensity of CD54 per cell. Thus, the average value of the CD54 intensity at each compound concentration is plotted on the graph as points 182. Using the plotted points, a simple curve 170 can be generated, as shown. This simple curve represents the dose response curve. Although FIGS. 7A-7C are shown as graphs with points plotted thereon, it is appreciated that step 164, as well as steps 166 and 170, discussed below, can be performed in a computer device using computer software or the like.

In step 166, the dose response curves generated in step 164 are qualified to determine a reaction of the cellular targets to the increasing concentrations of the compound. In one embodiment, qualifying the dose response curve can be accomplished by categorizing the curve as “increasing,” “decreasing,” or “flat.”

For example, to determine the curve characterization of a dose response curve, the dose response curve can be compared to a predefined range, such as range 184 in FIG. 7A, defined as the portion of the graph between upper and lower range limits 186 and 188. The predefined range can be determined using a standard deviation value, a percentage of total range, or a percentage of highest value. The predefined range 184 can be determined using other standards, as well. In the experimental results, discussed below, the predefined range was determined using a percentage of the highest value for the curve.

If the entirety of the curve falls within the predefined range, as is the case with dose response curve 172 of FIG. 7A, the curve can be characterized as “flat.”

If the entirety of the curve does not fall within predefined range 184, an evaluation can be made of the relationship between the lowest concentration of the compound and the highest value of the target. If the highest cellular target value occurs at the lowest compound concentration, the curve can be characterized as “decreasing.” For example, in FIG. 7B the highest target value corresponds to data point 182a, which occurs at the lowest compound concentration. As such, dose response curve 172 can be characterized as “decreasing.”

In contrast, if the lowest cellular target value occurs at the lowest compound concentration the curve can be characterized as “increasing.” For example, in FIG. 7C the lowest target value corresponds to data point 182b, which occurs at the lowest compound concentration. As such, dose response curve 174 can be characterized as “increasing.”

If the entirety of the curve does not fall within predefined range 184 and the lowest or highest cellular target values does not occur at the highest or lowest compound concentration, it may be unclear how the treated cells are reacting to the compound, and the particular cellular target might need to be discarded. For some cellular targets, these biphasic curves may be indicative of toxicity and more testing may be desired.

In step 170, a decision matrix is applied to the qualified dose response of one or more of the measured parameters to predict skin sensitization. The qualified dose response of any of the measured cellular targets can be used. For example, the cellular targets used in the decision matrix can be selected from the gross markers, specific markers and/or the functional response of the cells. Using the selected properties, a decision matrix can be constructed based upon the expected behavior of the cell type in response to the compound used. The dose response curve behavior (e.g., “increasing,” “flat,” or “decreasing”) of the measured cellular properties can then be used to determine the prediction of sensitizing capability of the compound. In some embodiments, the decision matrix can also be used to determine if the compound is toxic.

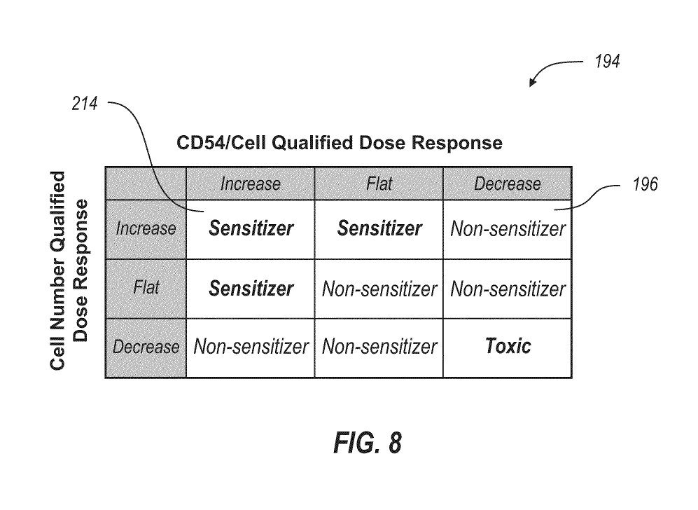

FIG. 8 depicts an example of a decision matrix 194 that predicts if a compound is a sensitizer based on the cell number target response of the immune effector cells compared with the CD54 intensity per cell target response. In decision matrix 194, the columns represent the possible CD54 intensity per cell qualified dose response and the rows represent the possible cell number qualified dose response. To determine a predicted skin sensitivity of a tested compound, one simply finds the matrix location corresponding to both of the qualified dose responses determined for the tested compound, and the entry within that matrix location identifies the predicted skin sensitivity of the compound. For example, if the qualified response of the treated immune effector cells was categorized as “increasing” in cell numbers and “decreasing” in CD54 intensity per cell, the corresponding matrix location in decision matrix 194 would be matrix location 196, which indicates that the corresponding compound would be predicted to be a non-sensitizer.

Although cell number and CD54 intensity responses are used in decision matrix 194, any of the other measured cellular targets can alternatively be used. For any of the cellular targets, the entry for each position in the decision matrix can be initially determined based upon the expected behavior of the immune effector cell type in response to the compound used. In addition, decision matrices can be designed that use more than two types of cellular targets as variables, again based upon the expected behavior of the immune effector cell type in response to the compound used.

### Testing Information

Test data and results are now given. Both known skin sensitizers and known non-skin sensitizers were used for testing. Table 1 lists the compounds used for testing; Table 1a includes the known skin sensitizer compounds used and Table 1b includes the known skin non-sensitizer compounds used. The characterization of each compound as a skin sensitizer or a skin non-sensitizer is based on a previous European Union project, entitled “Sens-it-iv”, that determined which of the compounds were skin sensitizers (European Union, 6th Framework, Novel Testing Strategies for In Vitro Assessment of Allergens LSHB-CT-2005-018681).

TABLE 1
Compounds Used for Skin Sensitizing Prediction Assay

a. Known Skin Sensitizers
2-aminophenol
2bromo-2bromomethylglutaronitrile
2hydroxyethylacrylate
Geraniol
Glyoxal
Hexylcinnaldehyde
Sodium Lauryl Sulfate
b. Known Skin Non-sensitizers
Acetone
Benzylaldehyde
Butanol
Diethyl Phthalate
DMF
DMSO
Ethanol
Ethyl Vanillin
Ethanol/Diethyl Phthalate
Glycerol
Isopropanol
Lactic Acid
Octanoic Acid
Phenylethylenediamine
Propylene Glycol
Salicylic Acid
Tween 80

An example protocol that was used to culture, treat, and stain the cell cultures during execution of the steps of the inventive methods discussed herein is now given. It is appreciated that the protocol discussed below is exemplary only and that other protocols can also be used.

The protocol is divided into two parts, the first directed to treatment and immunofluorescence of cellular targets that can be obtained using a phenotypic approach and the second directed to treatment and staining of cellular targets that can only be obtained using a chemotactic approach.

In both protocols, 96-well microplates, such as well plate 130 shown in FIG. 3, can be used for the assay. A plate set up or map, as discussed above, can be used to treat the drugs in the wells. For example, a plate can be assigned to be treated with the compounds in the microplate according to the example set up as shown in FIG. 4. It will be appreciated that other plate sizes and set ups can also be used.

The compound solutions can be prepared in appropriate solutes. Because of the number of compounds tested per plate and the requisite dilutions, making a master plate of individual wells of the 2× compound solutions that correspond to the plate map is recommended, although not required.

Treatment And Immunofluorescence For Phenotypic Analysis.

For treatment, staining, and immunofluorescence for the phenotypic approach, approximately 10,000 U937 cells can be placed in 100 μl of RPMI-1640 complete media per well in a collagen-I coated 96-well plate and incubated for 16-24 hours at 37° C. in 5% CO₂. To reduce variation between wells when using multiple plates, the plates can be spread in the incubator instead of stacked.

The compounds for which skin sensitivity is desired can then be introduced to the immune effector cells. For each concentration level of each compound, 100 μl of the 2× concentrated compound solution can be added to the corresponding wells. Similarly, 100 μl of vehicle solution can be added to the vehicle control wells and the 2× solution of the negative and positive controls can be added to the negative and positive control wells, respectively. The plate can then be incubated for 24 hours at 37° C. in 5% CO₂.

After incubation, the plate can be centrifuged for 5 minutes at 125×g. The media can then be aspirated from the wells after which 50 μl of pre-warmed (e.g., to 37° C.) 4% paraformaldehyde can be added to each well. The plate can then be incubated at room temperature for 15 minutes.

After incubation, the plate can be centrifuged for 5 minutes at 125×g. The paraformaldehyde can then be aspirated from the plate and the wells rinsed twice with 1× Phosphate Buffered Saline (PBS).

50 μl of 0.1% Triton X-100 dissolved in 1×PBS can be added to each well. The plate can then be incubated at room temperature for 10 minutes. The plate can be centrifuged for five minutes at 125×g, after which the liquid can be aspirated from the plate.

A primary antibody solution can be prepared as follows: For each 96-well microtiter plate, 6 ml of PBS are added to a 15 ml conical tube. To this, 12 μl of a primary antibody against CD54 (e.g., ICAM-1) are added. The resulting solution in the tube can be briefly vortexed (e.g., for less than 10 seconds).

After aspiration of the liquid, discussed above, 50 μl of the prepared primary antibody solution can be added to each well and the plate can be incubated at room temperature for one hour.

After the primary antibody solution has been added and the plate incubated, the plate can be centrifuged for 5 minutes at 125×g. The primary antibody solution can then be aspirated from the plate, and the wells rinsed twice with 1× Phosphate Buffered Saline (PBS).

A secondary staining solution can be prepared as follows. For each 96-well microtiter plate, 6 ml of PBS can be added to a 15 ml conical tube. To this, 6 μl of a secondary antibody can be added against the primary antibody that recognized CD54, along with 3 μl of Hoechst and 50 μl of the whole cell dye. The resulting solution in the tube can be briefly vortexed (e.g., for less than 10 seconds).

After the primary antibody solution has been aspirated from the plate and the wells rinsed, as discussed above, 50 μl of the secondary staining solution can be added to each well and the plate can be incubated at room temperature for 30 minutes, protected from light (e.g., wrapped in aluminum foil).

The plate can then be centrifuged for 5 minutes at 125×g, after which the liquid can be aspirated from the plate and the wells rinsed twice with 1×PBS.

A final volume of 150 μl of 1×PBS can be added to each well and the plate can be sealed. Image acquisition can then be performed on the plate using an imaging system such as a high-content screening (HCS) instrument using appropriate image analysis software.

Treatment And Staining For Chemotactic Analysis.

For treatment, staining, and immunofluorescence for the chemotactic approach, approximately 30,000 U937 cells can be placed in 100 μl of RPMI-1640 complete media per well in an uncoated 96-well plate and incubated for two hours at 37° C. in 5% CO₂. To reduce variation between wells when using multiple plates, the plates can be spread in the incubator instead of stacked.

The compounds for which skin sensitivity is desired can then be introduced to the immune effector cells. For each compound solution, 100 μl of the 2× concentrated compound solution can be added to the corresponding wells. Similarly, 100 μl of vehicle solution can be added to the vehicle control wells and the 2× solution of the negative and positive controls can be added to the negative and positive control wells, respectively. The plate can then be incubated for 24 hours at 37° C. in 5% CO₂.

After incubation, 50 μl of a 5× solution of a live cell reagent in 1×PBS can be added to each well, after which the plate can be incubated for 45 minutes at 37° C. in 5% CO₂.

The plate containing the treated cells can be carefully removed from the incubator so as not to jostle the cells from their settled state. 150 μl of the media can be removed from each well, and the cells in the remaining 100 μl of media can be resuspended.

A chemotactic solution of SDF-1 can be prepared at a concentration of 100 ng/ml. 200 μl of the chemotactic solution can be added to each well of a 96-well transwell plate with 8 μm pores. The transwell inserts can then be positioned within the wells so that no air bubbles are caught between the membrane of the inserts and the media. 50 μl of the resuspended cells from the original plate can then be added to the top of the transwell inserts.

The transwell plate can then be incubated for 2-6 hours at 37° C. in 5% CO₂ until migration has been achieved in the positive control wells. Image acquisition can then be performed using an imaging system such as a high-content screening (HCS) instrument using appropriate image analysis software.

### Test Results

The protocols discussed above were employed in conducting an assay on the twenty four chemical compounds listed in Table 1. The overall results are shown in Table 2.

TABLE 2
Assay Results

| | | |
|---|---|---|
| | Cell Number | |
| Compound | Result | Outcome |

a. Known Skin Sensitizers

| | | | |
|---|---|---|---|
| | CD54/Cell | | |
| | Result | | |
| 2-aminophenol | Increase | Increase | Sensitizer |
| 2bromo- | Decrease | Decrease | Toxic* |
| 2bromomethylglutaronitrile | | | |
| 2hydroxyethylacrylate | Increase | Increase | Sensitizer |
| Geraniol | Increase | Increase | Sensitizer |
| Glyoxal | Increase | Decrease | Sensitizer |
| Hexylcinnaldehyde | Increase | Increase | Sensitizer |
| Sodium Lauryl Sulfate | Increase | Increase | Sensitizer |

b. Known Skin Non-sensitizers

| | | | |
|---|---|---|---|
| | CD54/Cell | | |
| | Number | | |
| | Result | | |
| Acetone | Increase | Decrease | Sensitizer |
| Benzylaldehyde | Increase | Increase | Sensitizer |
| Butanol | Flat | Increase | Non-sensitizer |
| Diethyl Phthalate | Decrease | Decrease | Toxic* |
| DMF | Increase | Increase | Sensitizer |
| DMSO | Decrease | Increase | Non-sensitizer |
| Ethanol | Decrease | Increase | Non-sensitizer |
| Ethyl Vanillin | Decrease | Decrease | Toxic* |
| Ethanol/Diethyl Phthalate | Decrease | Decrease | Toxic* |
| Glycerol | Flat | Increase | Non-sensitizer |
| Isopropanol | Decrease | Increase | Non-sensitizer |
| Lactic Acid | Decrease | Decrease | Toxic* |
| Octanoic Acid | Increase | Increase | Sensitizer |
| Phenylethylenediamine | Decrease | Increase | Non-sensitizer |
| Propylene Glycol | Decrease | Flat | Non-sensitizer |
| Salicylic Acid | Decrease | Decrease | Toxic* |
| Tween 80 | Decrease | Increase | Non-sensitizer |

*at concentrations tested

A 96-well plate similar to well plate 130 shown in FIG. 3 was used for the assay. The plate map 134 shown in FIG. 4 was used for the 96-well plate.

The test protocol was optimized for use with U937 cells. To maintain U937 cells, supplier recommendations were adhered to. Cells were cultured in RPMI-1640 media supplemented with 2 mM glutamine, penicillin-streptomycin, and 10% fetal bovine serum. Cells were fed by replacing 90% of the media when a concentration of one million cells/ml of media was reached. Cells were used at a passage ≦20.

Cells were harvested by centrifugation, and concentrated into complete media so that the density was at one million cells/ml. 100 μl of the cell suspension was added to each well of the 96-well microplate to achieve 10,000 cells/well. Cells were incubated overnight at 37° C. in 5% CO₂ before compound treatment and then incubated again for 24 hours.

After the incubation with each of the compounds, the protocol discussed above was used to culture, treat, and stain the cell cultures. The following specific products were used during testing of each compound:
Human ICAM-1/CD54 MAb (Clone BRIG-11), Mouse IgG1 manufactured by R&D Systems was used as the primary antibody ICAM against CD54;
Goat anti-Mouse DyLight 650, manufactured by ThermoFisher Scientific was used as the secondary antibody;
Cellomics Whole Cell Stain Green dye, manufactured by ThermoFisher Scientific was used as the whole cell dye;
Live Cell Green 8410000 reagent, manufactured by ThermoFisher Scientific was used as the live cell reagent;
Recombinant Human/Rhesus Macaque/Feline CXCL12/SDF-1a, CF, manufactured by R&D Systems was used as the SDF-1 solution;
BD Falcon™ Transwell Multiwell 96 well insert system, manufactured by BD was used as the transwell plate.

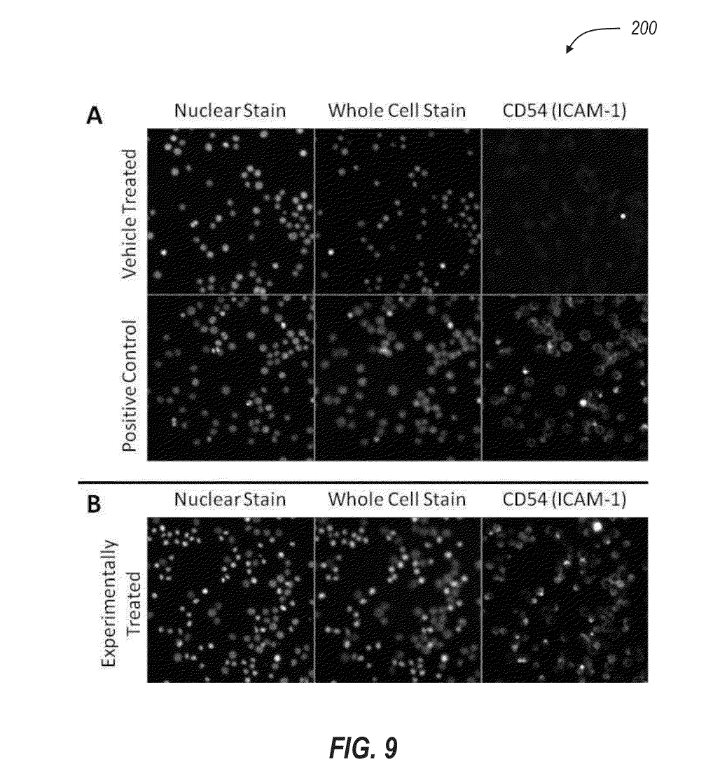

For each compound, cell images were obtained using the Thermo Scientific® ArrayScan® VTI HCS Reader, manufactured by Cellomics Inc. Representative samples 200 of the cell images obtained during the assay are shown in FIG. 9.

The data for each compound were normalized and dose response curves generated, as discussed above. The number of cells and the CD54 intensity per cell were used as the cellular targets used for the dose response curves. The dose response curves of each compound were qualified in the manner discussed above, and the decision matrix shown in FIG. 8 was used to then determine a prediction for the particular compound.

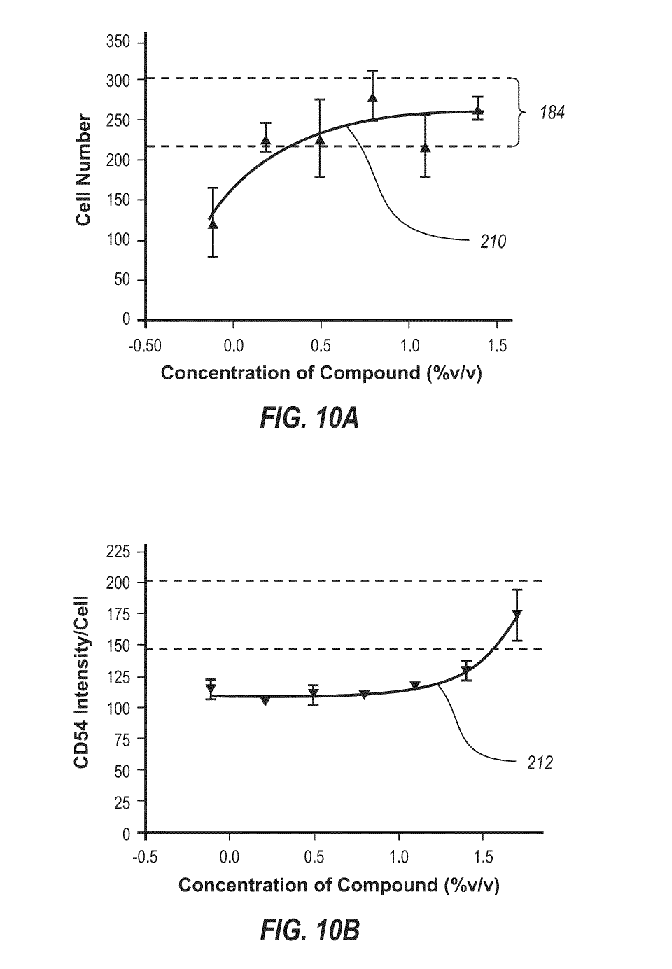

Using hexylcinnaldehyde as an example from Table 1, the dose response curves 210 and 212 shown in FIGS. 10A and 10B were obtained. Both of the dose response curves 210 and 212 were characterized as “increasing” using the steps discussed above. As noted above, the predefined range 184 was determined using a percentage of the highest value for the curve. Returning to FIG. 8, matrix position 214 of decision matrix 194 was used, because the dose response curves of both of the measured cellular targets were characterized as “increasing”. Therefore, from the results of the test, hexylcinnaldehyde was correctly predicted to be a skin sensitizer, as indicated in Table 2.

### Analysis of Test Results

As shown in Table 2, the assay yielded an 67% accuracy rate and 100% sensitivity for the skin sensitivity predictions. That is, of the known skin-sensitizers tested, the assay correctly predicted that all of them were skin sensitizers or toxic.

The results did not predict the skin non-sensitizers with as much accuracy. While most of the known skin non-sensitizers were correctly predicted to be skin non-sensitizers, a few of the known skin non-sensitizers were incorrectly predicted to be skin sensitizers. However, all of the chemicals that were predicted to be skin non-sensitizers were indeed skin non-sensitizers. Thus, although a definitive prediction of skin sensitization was not obtained, one of skin non-sensitization was. That is, because all of the chemicals that were predicted to be skin non-sensitizers were indeed skin non-sensitizers, one can deduce that if the outcome of the assay indicates that a compound is a non-sensitizer, then the compound indeed is a non-sensitizer.

This information can be used to reduce the number of compounds required to be animal-tested for skin sensitivity. For example, one can use the assay as a preliminary screening to remove from further testing those compounds that are predicted to be skin non-sensitizers. Additional testing can then be performed on the chemicals that are predicted to be skin sensitizers or toxic. By eliminating the predicted skin non-sensitizers from further testing, a great deal of time, money, and effort can be saved, even if the other chemicals may need to undergo further testing.

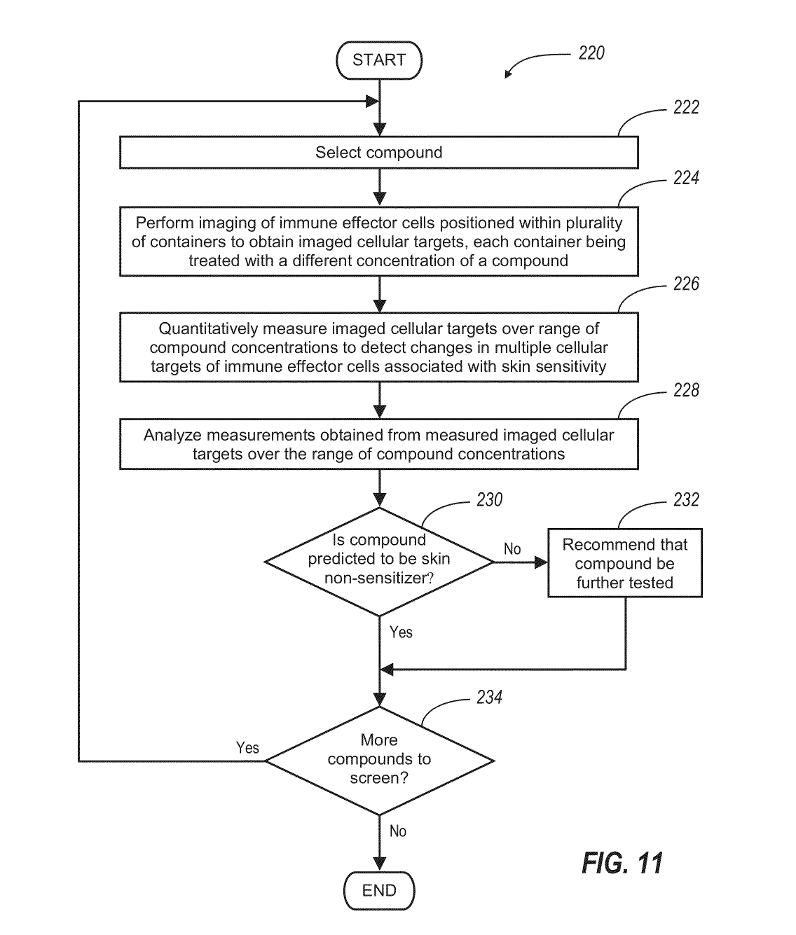

FIG. 11 illustrates an embodiment of a method 220 of screening a plurality of compounds for skin sensitivity. In step 222, a compound of interest is selected. In steps 224, 226, and 228, the compound is tested, in the manner discussed above, to determine if the compound is predicted to be a skin sensitizer or a skin non-sensitizer. In step 230, if the compound is not predicted to be a skin non-sensitizer (i.e., the compound is predicted to be a sensitizer or toxic), then the compound is recommended to be further tested in step 232. Otherwise, step 232 is skipped. The recommendation can be done, e.g., by setting a flag in software. In step 234, if there are more compounds to screen, the method is repeated, starting again at step 222. In one embodiment, the method further includes performing the further testing for the compounds recommended for the further testing.

Besides being useful as a screening tool, further testing will likely determine further cellular target combinations that may be even more accurate predictors of skin sensitivity than those used in present testing.

Many benefits to skin sensitivity testing over present methods are obtained by using embodiments of the present invention. Some of these include:
requires no animals;
replicates the in vivo effects of a skin sensitizer by measuring functional changes;
is less costly and less labor intensive than in vivo approaches;
determines sensitization at different concentrations of the compound;
can indicate if a compound is toxic at tested concentrations;
can test many compounds concurrently, yielding a high throughput.

Although discussion herein has been directed to determining the sensitivity of skin to tested compounds using the methods presented herein, it is appreciated that other types of sensitivity can also be predicted using the methods. For example, sensitivity of other body tissue or organs to tested compounds can also be predicted. For example, sensitivity of the throat, the stomach, the lungs and associated airways, the eyes, etc. can also be predicted by imaging and analyzing corresponding cells according to the methods presented herein.

Furthermore, sensitivity potential of a compound used in conjunction with non-chemical insults, such as UV-radiation, can also be determined using the methods presented herein.

The present invention may be embodied in other specific forms without departing from its spirit or essential characteristics. Accordingly, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method of predicting whether a compound is a skin sensitizer, the method comprising:
imaging immune effector cells positioned within a plurality of containers to obtain imaged cellular targets, each container being treated with a different concentration of a compound;
quantitatively measuring the imaged cellular targets over a range of concentrations of the compound to detect changes in multiple cellular targets of the immune effector cells associated with skin sensitivity; and
analyzing measurements obtained from the measured imaged cellular targets over the range of concentrations of the compound to determine whether the compound is a skin sensitizer.

2. The method recited in claim 1, wherein the imaging of the immune effector cells is performed using a quantitative high-content cell imaging system.

3. The method recited in claim 1, wherein imaging immune effector cells comprises:
using a phenotypic approach on a first portion of the treated immune effector cells; and
using a chemotactic approach on a second portion of the treated immune effector cells.

4. The method recited in claim 1, wherein analyzing measurements obtained from the measured imaged cellular targets over the range of concentrations to determine whether the compound is a skin sensitizer comprises:
determining data normalization values for each cellular target;
determining a representative value for each cellular target at each concentration of the compound;
generating a dose response curve for each cellular target based on the representative values of the cellular target;
qualifying a dose response of each cellular target based on the dose response curve of the cellular target to determine a reaction of the cellular target to increasing concentrations of the compound; and
applying a decision matrix to the qualified dose responses.

5. The method recited in claim 4, wherein determining data normalization values for each cellular target comprises subtracting a baseline response of vehicle-treated cells from measurements of the measured imaged cellular targets over the range of concentrations of the compound.

6. The method recited in claim 4, wherein determining the representative value for each cellular target at each concentration of the compound comprises determining an average value for each cellular target at each concentration of the compound.

7. The method recited in claim 4, wherein generating a dose response curve for each cellular target comprises:
plotting the representative value of the cellular target corresponding to each concentration of the compound; and
fitting a curve to the plotted values.

8. The method recited in claim 4, wherein qualifying the dose response of each cellular target comprises characterizing the dose response curve of the cellular target as one of: increasing, decreasing, and flat.

9. The method recited in claim 8, wherein characterizing the dose response curve comprises:
characterizing the dose response curve as flat if all of the dose response curve is positioned within a predefined range;
otherwise:
characterizing the dose response curve as decreasing if a highest value of the dose response curve occurs at a lowest concentration of the compound; or
characterizing the dose response curve as increasing if a lowest value of the dose response curve occurs at a lowest concentration of the compound.

10. The method recited in claim 4, wherein applying the decision matrix comprises determining whether the compound is a skin sensitizer based upon expected behavior of the qualified dose responses of two or more cellular targets.

11. The method recited in claim 4, wherein applying the decision matrix comprises predicting that the compound is one of: a skin sensitizer, a skin non-sensitizer, or toxic to the skin.

12. The method recited in claim 1, wherein the imaged cellular targets comprise one or more of: a gross marker, a specific marker, and a functional outcome.

13. The method recited in claim 1, wherein the imaged cellular targets includes at least one of cell trafficking and cell motility imaged using a chemotactic approach.

14. (canceled)

15. A method of screening a plurality of compounds for skin sensitivity, the method comprising:
for each compound:
imaging immune effector cells positioned within a plurality of containers to obtain imaged cellular targets, each container being treated with a different concentration of the compound, the imaging being performed using a quantitative high-content cell imaging system;
quantitatively measuring the imaged cellular targets over a range of concentrations of the compound to detect changes in multiple cellular targets of the immune effector cells associated with skin sensitivity;
analyzing measurements obtained from the measured imaged cellular targets over the range of concentrations of the compound to determine if the compound is predicted to be a non-skin sensitizer; and
recommending that the compound be further tested if the compound is not predicted to be a non-skin sensitizer.

16. The method recited in claim 15, further comprising performing further testing for skin sensitivity on the compounds that are not predicted to be non-skin sensitizers.

17-18. (canceled)

19. The method recited in claim 1, wherein the immune effector cells comprise U937 cells.

20. The method recited in claim 1, wherein analyzing measurements obtained from the measured imaged cellular targets over the range of concentrations of the compound is also performed to determine whether the compound is toxic to the skin.

21. A method of predicting whether a compound is a skin sensitizer, the method comprising:
culturing immune effector cells positioned within a plurality of containers;
treating each container with a different concentration of a compound;
staining the cells in the containers with fluorescent materials;
transferring the stained cells to a chemotaxis plate;
applying chemokine to the stained cells in the chemotaxis plate; and
acquiring images from the stained cells after the chemokine has been applied thereto;
quantitatively measuring the imaged cellular targets over a range of concentrations of the compound to detect changes in multiple cellular targets of the immune effector cells associated with skin sensitivity; and
analyzing measurements obtained from the measured imaged cellular targets over the range of concentrations of the compound to determine whether the compound is a skin sensitizer.

22. The method recited in claim 21, wherein analyzing measurements obtained from the measured imaged cellular targets comprises:
determining data normalization values for each cellular target;
determining a representative value for each cellular target at each concentration of the compound;
generating a dose response curve for each cellular target based on the representative values of the cellular target;
qualifying a dose response of each cellular target based on the dose response curve of the cellular target to determine a reaction of the cellular target to increasing concentrations of the compound; and
applying a decision matrix to the qualified dose responses.

23. The method recited in claim 22, wherein generating the dose response curve for each cellular target comprises:
plotting the representative value of the cellular target corresponding to each concentration of the compound; and
fitting a curve to the plotted values.
